(19) United States
(12) Patent Application Publication
(10) Pub. No.: US 2001/0038844 A1
(43) Pub. Date: Nov. 8, 2001

(54) METHODS AND VACCINES FOR PROTECTION AGAINST CAMPYLOBACTER INFECTIONS

(76) Inventor: IRVING NACHAMKIN, NEWTON SQUARE, PA (US)

Correspondence Address: JANE MASSEY LICATA, LAW OFFICES OF JANE MASSEY LICATA, 66 E MAIN STREET, MARLTON, NJ, 08053

(21) Appl. No.: 09/300,975

(22) Filed: Apr. 28, 1999

Related U.S. Application Data
(60) Provisional application No. 60/084,170, filed on May 4, 1998.

(51) Int. Cl. A61K 39/02

(52) U.S. Cl. 424/200.1; 424/234.1

(57) ABSTRACT

Methods and vaccines for protection of animals against Campylobacter infection are provided.

This application claims the benefit of U.S. provisional patent application No. 60/084,170, filed May 4, 1998.

### INTRODUCTION

This invention was supported in part by funds from the U.S. government (USDA Grant No. 95-37201-2980) and the U.S. government may therefore have certain rights in the invention

### BACKGROUND OF THE INVENTION

Campylobacter jejuni is a major cause of gastrointestinal infection in man and is estimated to be the most common cause of sporadic bacterial diarrheal illness with approximately 2 million cases per year in the United States alone (Tauxe, R. V. 1992. Campylobacter jejuni: Current Status and Future Trends, pp. 9-19). C. jejuni typically causes an acute enterocolitis accompanied by fever and abdominal cramping lasting 3 to 5 days. Although complications are infrequent, Campylobacter infection may be confused with acute appendicitis resulting in unnecessary surgery, and may also cause serious extraintestinal infections (Nachamkin, I. 1993. Curr. Opin. Infect. Dis. 6:72-76). There is now substantial evidence that Campylobacter is a major cause of a serious paralytic illness, Guillain-Barre Syndrome (GBS; Mishu, B. and M. J. Blaser. 1993. Clin. Infect. Dis. 17:104-108), including both demyelinating and axonal forms of the disease (McKann, G. M. et al. 1993. Ann. Neurol. 33:333-342). Although rare, deaths have been associated with Campylobacter infection. The morbidity due to the disease is significant and results in substantial economic costs (Tauxe, R. V. 1992. Campylobacter jejuni: Current Status and Future Trends, pp. 9-19).

Most of the cases of Campylobacter infection in the U.S. occur sporadically, although common source outbreaks are also seen. Campylobacter infection is essentially a food-borne illness acquired by ingestion of contaminated water, milk, and/or food products. A number of animal reservoirs serve as vehicles for transmission of Campylobacter, with poultry being the single most important vehicle for transmission of human infection (Stern, N. J. 1992. Campylobacter jejuni: Current Status and Future Trends, pp. 49-60). Other reservoirs for the organism include ducks, turkeys, cattle, goats, sheep, and swine.

While the mechanism by which C. jejuni colonizes chickens remains unclear, intact motile flagella (Nachamkin, I. et al. 1993. Appl. Environ. Microbiol. 59:1269-1273) and the type of flagellin expressed (Wassenaar, T. M. et al. 1993. J. Gen. Microbiol. 139:1171-1175) are known to be important factors. C. jejuni are motile by means of a single polar, unsheathed flagellum at one or both ends of the bacterium. Two genes, flaA and flaB are involved in the expression of the flagellar filament (primarily flaA) and are present in both C. jejuni (Fisher, S. H. and I. Nachamkin. 1991. Mol. Microbiol. 5:1151-1158; Nuijten, P. J. et al. 1990. J. Biol. Chem. 265:17798-17804) and Campylobacter coli (Guerry, P. et al. 1990. J. Bacteriol. 172:1853-1860). Using a series of flagellar mutants, it has been shown that only fully motile organisms with complete flagella colonized three-day old chicks (Nachamkin, I. et al. 1993. Appl. Environ. Microbiol. 59:1269-1273); non-motile or partially motile mutants did not colonize the chick caecum. FlaA mutants of C. jejuni were shown to colonize chicks with much less efficiency (100- to 1000-fold less) than the wild type strain (Wassenaar, T. M. et al. 1993. J. Gen. Microbiol. 139:1171-1175). In addition to flagella, other cellular components may be necessary for colonization (Hugdahl, M. B. et al. 1988. Infect. Immun. 56:1560-1566; Meinersmann, R. J. et al. 1991. Am. J. Vet. Res. 52:1518-1522; Meinersmann, R. J. et al. 1990. Curr. Microbiol. 21:17-21).

Studies have indicated that the humoral immune response raised against Campylobacter can be highly variable. Some studies have indicated that humoral immune responses against Campylobacter flagellin may be important in preventing colonization of the chicken gastrointestinal tract (Cawthraw, S. et al. 1994. Avian Dis. 38:341-349; Myszewski, M. A. and N. J. Stern. 1990. Avian Dis. 34:588-594; Stern, N. J. et al. 1990. Avian Dis. 34:595-601). A recent report by Khoury and Meinersmann (1995. Avian Dis. 39:812-820) demonstrated that a hybrid protein comprised of Campylobacter jejuni flagellin fused to the beta-subunit of the labile toxin of E. coli decreased colonization following oral dosing in chickens. The number of birds that were colonized at 5 weeks of age was significantly lower in the vaccinated group as compared to controls. However, the degree of protection afforded by the vaccination strategy was not quantified.

Other studies with similar antigens, however, have failed to demonstrate reproducible effects. For example, a whole killed Campylobacter jejuni vaccine as well as purified flagellin antigen was tested in young chicks. Day old chicks immunized with purified flagellin (in adjuvant, 4 doses) via intraperitoneal injection failed to mount an immune response to flagellin. Further, animals vaccinated with two doses of flagellin either intraperitoneally or combined with oral dosing at 16 and 29 days of age and rechallenged with homologous live organisms failed to show any reduction in cecal colonization. Animals vaccinated with a combination of flagellin and whole cells by the intraperitoneal route (first dose) and oral route (second dose), however, did show a significant reduction in cecal counts, which was attributed to antibodies formed against whole cell antigens (Widders, P. R. et al. 1996. Br. Poul. Sci. 37:765-778). Accordingly, there is a need for compositions capable of producing a measurable, antibody-specific response to Campylobacter antigen.

A number of avirulent strains of Salmonella capable of eliciting mucosal immune responses in chickens have been described. For example, S. typhimurium delta-cya-delta-crp mutant was developed by Curtiss and Kelly (Curtiss, R. and S. M. Kelley. 1987. Infect. Immun. 55:3035-3044) and further modified by Galen and colleagues (Galen, J. E. et al. 1990. Gene 94:29-35). Also see U.S. Pat. No. 5,424,065 disclosing development of another mutant strain of Salmonella that has a mutation in a phop gene, as well as use of these avirulent mutant organisms as components of vaccines against Salmonella typhimurium. S. typhimurium X3985 delat-cya-delta-crp elicited significant protection against rechallenge with a virulent strain of Salmonella in chickens, a response that was shown to be dose and strain dependent (Hassan, J. O. and R. Curtiss. 1990. Res. Microbiol. 141:839-850). However, as taught by Curtiss R. et al., U.S. Pat. No. 5,424,065, successful application of these Salmonella mutants is not assured in all situations but instead is dependent on the host, the bacterial species, and the route of immunization. Salmonella mutants have also been used as vectors to express foreign antigens from Streptococcus mutants, S. sobrinus (Doggett, T. A. et al. 1993. Infect. Immun. 61:1859-1966; Jagusztyn-Krynicka, E. K. et al. 1993. Infect. Immun. 61:1004-1015; Redman, T. K. et al. 1995. Infect. Immun. 63:2004-2011; Redman, T. K. et al. 1996. Vaccine 14:868-878), S. equi, Bordetella avium (Gentry-Weeks, C. R. et al. 1992. J. Bacteriol. 174:7729-7742), B. pertussis, Mycobacterium (Curtiss, R. et al. 1990. Res. Microbiol. 141:797-805), hepatitis B virus (Schodel, F. et al. 1994. Infect. Immun. 62:1669-1676), E. coli heat labile toxin-viral fusion proteins (Smerdou, C. et al. 1996. Virus Res. 41:1-9), and tetanus toxin fragment C (Karem, K. L. et al. 1995. Infect. Immun. 63:4557-4563).

An avirulent Salmonella strain has also been used to express the 72DZ/92 gene of Campylobacter jejuni (Pawelec, D. et al. 1997. FEMS Immunology and Medical Microbiology 19:137-140). This gene is demonstrated to encode immunopositive proteins that through sequence homology appears to belong to family 3 solute binding proteins, components of the ABC transporter system. These proteins are referred to as CjaA, CjaB and CjaC. Pawelec et al. suggest that construction of S. typhimurium strains expressing C. jejuni genes encoding a protective C. jejuni antigen such as CjaA, CjaC, LPS, MOMP, flagella or a flagellin subunit, might provide an effective method of obtaining chicken vaccines against both enteropathogens. However, no experiments demonstrating efficacy of such a vaccine are provided. Further, there is no demonstration of the ability to express any other antigens of C. jejuni in an avirulent Salmonella strain.

It has now been found that an avirulent Salmonella vector expressing Campylobacter antigen provides an effective live, oral vaccine against Campylobacter infection.

### SUMMARY OF THE INVENTION

The present invention provides compositions and methods for protection of animals against Campylobacter infection. An object of the present invention is to provide a recombinant vaccine for immunization of animals against Campylobacter which comprises an avirulent strain of Salmonella that expresses a Campylobacter antigen. The antigen can be a Campylobacter flagellar protein or a fragment of the flagellar protein.

Another object of the present invention is to provide a method of protecting animals from infection by a strain of Campylobacter which comprises administration of the recombinant Salmonella vaccine.

Another object of the present invention is to provide a recombinant vaccine for immunization of animals against Campylobacter and Salmonella infections which comprises administration of the recombinant Salmonella vaccine.

Another object of the present invention is to provide a method of protecting animals from infection by strains of Campylobacter and Salmonella which comprises administration of the recombinant Salmonella vaccine.

### DETAILED DESCRIPTION OF THE INVENTION

The present invention provides compositions and methods that are useful in protecting animals against infection with Campylobacter. Vaccines of the present invention comprise an avirulent strain of Salmonella that expresses a Campylobacter antigen. In a preferred embodiment, a recombinant mutant Salmonella vector such as Salmonella asd, a mutation blocking production of aspartate beta-seialdehyde dehydrogenase, is used. The Salmonella asd mutant is preferred because these mutants have a requirement for diaminopimelic acid (DAP). DAP is a component of the cell wall of Salmonella and the organisms will not survive in the host animal without a source of DAP. DAP is not present in animal tissues, and, therefore, the asd mutant is avirulent because it cannot survive in animal tissues (Nakayama, K. et al. 1988. Bio/Technology 6:693-697). The Salmonella mutant known as asd is also a preferred embodiment because it does not rely on the use of antibiotic selection to maintain the plasmid, a commonly employed technique in vaccine development that can lead to transmission of antibiotic resistance in the host. The other advantages of the Salmonella mutants as a vector system include the well-established ability to express foreign antigens, as well as the use of the strain as a live vaccine to deliver antigens directly to the target tissue, gut lymphoid tissue. Therefore, this mutant strain of Salmonella is preferred for development of the recombinant Campylobacter protein expression system in the present invention. It is also preferred that the Campylobacter antigen or protein expressed by the Salmonella comprise Campylobacter flagellin protein or an antigenic fragment thereof.

The ability of the Salmonella vector to express the Campylobacter flagellin protein and to provide effective protection of animals against infection with Campylobacter was demonstrated. In these experiments, the flagellin gene, flaA, from C. jejuni IN1, cloned in pYA292 carrying the Salmonella asd gene was transduced into S. typhimurium _{X}4550. Two recombinants, UP271 and UP503 were selected for further study and synthesized an approximately 60 kDa protein that was slightly smaller than the predicted size. The slightly smaller size than the purified native protein is likely due to differences in post-translational modification of the protein that occurs in Campylobacter but not when the protein is expressed in the current expression system. Western blot analysis of whole cell lysates and outer membrane preparations showed that the 60 kDa protein was localized in the outer membrane fraction of UP271 while in UP503 the protein appeared to be present in the cytosolic fraction.

To test the efficacy of this vaccine, chicks were given two doses of oral vaccine at 4 and 6 days of age and challenged with approximately 10⁹ cfu of homologous C. jejuni at three weeks of age. The vaccine dosing schedule was developed to stimulate local immune responses as early in a chick's life as possible. Since an effective immune response must be operating within the first week of hatching, the time in a chick's life when the immune system matures, there must be a balance between the chick's age, vaccine dose, and time span of immunization and booster vaccination to obtain maximum protective immunity as early as possible to prevent or reduce colonization. This is especially important since the colonization dose of C. jejuni appears to decrease dramatically upon passage through chickens (Cawthraw, S. A. et al. 1996. Epidemicl. Infect. 117:213-215). Four separate experiments were performed using UP271 and three experiments were performed using UP503. For each experiment, there were 6 to 10 chicks for each control group and 8 to 10 chicks for each vaccine group.

For all chicks vaccinated with UP271 (4 experiments) there was a 2.261 mean log₁₀ reduction in cecal colony counts. For chicks vaccinated with UP503 (3 experiments), there was a 2.063 mean log₁₀ reduction in cecal colony counts. The differences between animals vaccinated with UP271 as opposed to UP503 were not statistically significant. For animals vaccinated with UP271, only 8/35 (22.9%) of the animals had less than a 1 log₁₀ reduction in counts. With UP503, only 7/29 (24.1%) had less than a 1 log₁₀ reduction in counts. In both cases, the animals with less than a 1 log₁₀ reduction in counts were from the second experimental group. The lack of complete protection afforded by the vaccine is likely due to the large challenge dose used in the experiments (10⁹ cfu). The dose needed to colonize chickens is actually much lower, in the range of 10² to 10⁶ cfu.

One of the four experimental groups vaccinated with UP271 were also used to assess the ability of UP271 to confer cross-protection using a heterologous strain of C. jejuni. A heterologous strain that was matched for O serotype but differed in flagellin gene type was used. C. jejuni flaA types can be distinguished by restriction fragment length polymorphism (RFLP) analysis with over 80 types having been described (Nachamkin, I. et al. 1993. J. Clin. Microbiol. 31:1531-1536; Nachamkin, I. et al. 1996. J. Clin. Microbiol. 34:277-281). The ability of the vaccine to confer crossprotection on strains with other than IN1, 04, or FlaA-16 flagellin was also examined. Using the same challenge protocol as before, chicks were vaccinated with UP271 and in addition to homologous rechallenge at 3 weeks of age were challenged with a heterologous strain, C. jejuni D3504 (04, FlaA-1). Animals challenged with the homologous strain showed a 3.87 mean log₁₀ reduction in cecal counts compared with controls. Animals challenged with the heterologous strain showed a 1.799 mean log₁₀ reduction compared with controls. While the heterologous strain conferred approximately 50% of the protection seen with the homologous strain, the differences between the two groups were not statistically significant.

In order to define the specificity of the vaccination response as being due to production of anti-flagellin antibodies, the immunogenicity of the vaccine was also examined. Serum was obtained from chicks at 4 days of age, prior to receiving vaccine (pre sera), at approximately 10 days after receiving vaccine (mid sera), and at 3 weeks of age (post sera), prior to rechallenge with live organisms. The serum was examined for the presence of IgG, IgA, and IgM antibodies directed against purified flagellin protein from the homologous vaccine-derived strain.

Chicks receiving vector alone did not show a significant rise in IgG, IgA or IgM anti-flagellin antibodies (P<0.05) Chicks receiving both UP271 and UP503 showed highly significant rises in serum IgG anti-flagellin antibodies. Compared with the mean values for pre sera IgG anti-flagellin antibodies in control animals, 66% of animals vaccinated with UP271 showed an increase in vaccine-induced anti-flagellin antibodies. In contrast, 100% of animals vaccinated with UP503 had a significant rise in IgG antibodies. IgA anti-flagellin antibodies developed above the mean pre sera levels in 64% of animals receiving UP271 and 57% of animals receiving UP503. Similarly, 64% and 36% of animals receiving UP271 and UP503, respectively, developed IgM anti-flagellin antibodies above the mean pre serum values.

The correlation between antibody level and the degree of protection varied and was dependent upon the antibody isotype analyzed. Except for IgG and IgM antibodies in the UP503 group, there was statistically significant correlation between antibody level and degree of protection. Considered together, these data indicate that protection induced against Campylobacter was antibody-mediated. These data are the first demonstration that a flagellin-specific immune response protected animals from infection and colonization of Campylobacter. It is also the first time that vaccination of chicks with Campylobacter flagellin resulted in significant homologous as well as heterologous protection against infection.

Accordingly, the compositions and methods of the present invention are useful in effective vaccination protocol for protection against Campylobacter infection by various strains of Campylobacter including, but not limited to, C. jejuni and C. coli. These compositions and methods are also useful in vaccinating animals against infection by Salmonella. The avirulent Salmonella mutant expressing the Campylobacter is administered in a pharmaceutically acceptable vehicle. Such vehicles for vaccines are well known in the art. It is preferred that vaccines of the present invention be administered orally, within the first few days of life of the animal. Further, the preferred dosing regimens include multiple doses of the oral vaccine within the first weeks of life. However, as will be obvious to those of skill in the art, other vaccination protocols which allow for optimal protection against infection in the species of interest can be used. In addition to chickens, other species known to be carriers of Campylobacter infection would be targeted including but not limited to ducks, turkeys, cattle, goats, sheep, swine and humans.

The following non-limiting examples are presented to better illustrate the claimed invention:

### EXAMPLES

### Example 1

Bacterial Culture

Bacterial strains and plasmids of E. coli and S. typhimurium were grown aerobically at 37° C. on LB agar or broth and when appropriate supplemented with diaminopimelic acid at 50 μg/ml. Campylobacter jejuni was grown on Campy CVA media in an atmosphere containing 5% O₂, 10% CO₂ and 85% N₂ at 42° C. For production of bacteriophage P22 lysates, S. typhimurium strains were grown in LB broth.

### Example 2

Expression of Campylobacter flagellin in S. typhimurium

General cloning techniques used have been described by Sambrook et al. (1989. Molecular Cloning: A Laboratory Manual, Cold Spring Harbor Press). Restriction enzymes were obtained from either Gibco BRL or Boehringer-Mannheim and used according to manufacturers' instructions. Oligonucleotides were synthesized using a PCR-Mate 391 Synthesizer.

The C. jejuni flaA gene (1.7 kB) was amplified by PCR using primers based on the DNA sequence previously published (Fisher, S. H. and I. Nachamkin. 1991. Mol. Microbiol. 5:1151-1158) except that EcoRI and BamHI restriction sites were added to each, respectively, forward or reverse oligonucleotide primer to assure the adequate gene orientation during DNA cloning. The forward primer, F-42, had the following sequence: 5′-CAAGTTCATGGATGGACTGA-3′ (SEQ ID NO:1). The reverse primer, 2R3, had the following sequence: 5′-CGGGATCCTACTGTAGTAATCTTAA-3′ (SEQ ID NO:2). Amplified DNA fragments were double digested with EcoRI/BamHI and cloned into the lac gene OF Pya292 SHUTTLE VECTOR dna CARRYING THE Salmonella asd gene complementing the synthesis of DAP (Nakayama, K. et al. 1988. Bio/Technology 6:693-697). Recombinant DNAs were transformed into E. coli _{X}6212 by electrotransformation and transformants were selected for DAP-independency using LB agar without DAP. Plasmid DNA isolated from E. coli _{X}6212 were electrotransformed into intermediate, restriction-negative, modification-positive S. typhimurium _{X}3730. The general transducing Salmonella phage P22HTint (Schmieger, H. 1972. Mol. Gen. Genet. 119:75-88) was used to move the flaA gene constructs into the final Salmonella recipient, S. typhimurium _{X}4550 as previously described (Gentry-Weeks, C. et al. 1992. J. Bacteriol 174:7729-7742; Karem, K. L. et al. 1995. Infect. Immun. 63:4557-4563). Transformants were selected on LB plates without DAP, and incubated at 37° C. Vaccine constructs were then characterized by PCR and Western Blot analysis.

### Example 3

Immunologic Assays

Campylobacter flagellin antigen was prepared as previously described (Nachamkin, I. and X. H. Young. 1992. J. Clin. Microbiol. 30:509-511). Outer membranes were prepared as sarkosyl insoluble fractions (Logan, S. M. and T. J. Trust. 1982. Infect. Immun. 38:898-906).

Western blots were performed as previously described with slight modifications (Nachamkin, I. and A. M. Hart. 1985. J. Clin. Microbiol. 21:33-38). Proteins were transferred onto Immobilon-P transfer sheets by electrotransfer. Membranes were blocked for 2 to 16 hours in blocking buffer (PBS, 1% BSA) followed by 1 to 2 hours of incubation at room temperature with primary polyclonal rabbit antisera raised against flagellin diluted 1:4000 or monoclonal mouse antibody F11-42 (Aguero-Rosenfeld, M. E. et al. 1990. Infect. Immun. 58:2214-2219) diluted 1:1000. Membranes were then washed three times for at least 20 minutes each with washing buffer (PBS-Tween, 0.1%) and incubated at room temperature for 1 hour with secondary antibodies (horseradish-peroxidase-linked goat anti-rabbit or goat anti-mouse antibody) and binding was detected with ECL Western blotting detection reagents.

Chicken serum antibodies directed against flagellin were measured using a modified ELISA assay (Aguero-Rosenfeld, M. E. et al. 1990. Infect. Immun. 58:2214-2219). Wells of round-bottomed Immunlon-2 microplates were coated overnight at 4° C. with 200 μl of C. jejuni flagellin suspension at a concentration of 10 μg/ml. Horseradish peroxidase-conjugated goat anti-chicken IgG, IgA, or IgM were used as the secondary antibody with 2,2′ azino bis 3-ethylbenzthiazoline 6-sulfonic acid diammonium substrate used to develop the reaction. All sera were tested at a dilution of 1:60 and run on the same day, in duplicate.

### Example 4

Production of Rabbit Antisera

New Zealand White rabbits that were free of C. jejuni were used to produce polyclonal immune sera. Two rabbits were used for immunization either to purified flagellin or whole C. jejuni cells. Rabbits were immunized subcutaneously with 10⁸ cfu of live C. jejuni IN1 strain or 150 μg of FlaA protein emulsified with MPL/TDM/CWS Adjuvant System (Sigma, St. Louis, Mo.) and boosted after three weeks with the same dose.

### Example 5

Vaccination of Animals Against Campylobacter jejuni

All chicks were unsexed, three day old White Leghorns (Truslow Farms, Chestertown, Md.) and were maintained in isolators provided with unlimited food and water. Prior to immunization, blood samples were obtained and C. jejuni cultures were performed on fecal material collected with cloacal swabs. For each experiment, the control group received S. typhimurium _{X}4550 (pYA292) and vaccine group received S. typhimurium _{X}4550 expressing flagellin (UP271 or UP503). Each bird in a group was inoculated orally with two doses, two days apart at 4 and 6 days of age, with 0.1 ml of S. typhimurium construct suspended in buffered saline gelatin at the density of 10⁹ cfu per ml and delivered directly into the crop with an animal feeding biomedical needle. Three weeks after the first oral immunization, all chicks were challenged orally in the same manner with 0.5 ml of C. jejuni (homologous or heterologous strain) suspension at a density of 1×10⁹ cfu per ml. Seven days after this inoculation, chicks were euthanized by intravenous injection of sodium pentobarbital. Blood was collected and the caecum contents removed, weighed, and sequential dilutions plated out on selective CVA Campy plates. Data for caecal infection were transformed by taking the log₁₀ of bacteria numbers per gram of caecal contents.

What is claimed is:

1. A recombinant vaccine for immunization of animals against Campylobacter infections comprising an avirulent Salmonella strain expressing a Campylobacter antigen.

2. The recombinant vaccine of claim 1 wherein the Campylobacter antigen comprises a Campylobacter flagella protein or fragment thereof.

3. A method of protecting animals from infection by a strain of Campylobacter comprising administering to an animal the recombinant vaccine of claim 1.

4. A recombinant vaccine for immunization of animals against Campylobacter and Salmonella infections comprising an avirulent Salmonella strain expressing a Campylobacter antigen.

5. The recombinant vaccine of claim 4 wherein the Campylobacter antigen comprises a Campylobacter flagella protein or fragment thereof.

6. A method of protecting animals from infection by strains of Campylobacter and Salmonella comprising administering to an animal the recombinant vaccine of claim 4.


SEQUENCE LISTING (2 sequences)

SEQ ID NO: 1 - DNA, 20 bp, Artificial Sequence
  Description of Artificial SequenceSynthetic
caagttcatggatggactga

SEQ ID NO: 2 - DNA, 25 bp, Artificial Sequence
  Description of Artificial SequenceSynthetic
cgggatcctactgtagtaatcttaa